(19) United States
(12) Patent Application Publication
(10) Pub. No.: US 2004/0204759 A1
(43) Pub. Date: Oct. 14, 2004

(54) Prosthesis with foldable flange

(76) Inventors: Eric Blom, Carmel, IN (US); Jobeth Seder, Carpinteria, CA (US)

Correspondence Address: Marvin E Jacobs, Koppel Jacobs Patrick & Heybl, Suite 215, 2151 Alesandro Drive, Ventura, CA, 93001, US

(21) Appl. No.: 10/487,612

(22) PCT Filed: Dec. 20, 2002
(86) PCT No.: PCT/US02/40932

Related U.S. Application Data
(60) Provisional application No. 60/344,592, filed on Dec. 28, 2001.

(51) Int. Cl. A61F 2/20; A61F 2/18

(52) U.S. Cl. 623/9; 623/10

(57) ABSTRACT

Insertion of a flange on a tubular prosthesis such as a voice prosthesis through a body opening such as a tracheoesophageal fistula is facilitated by forming a thinner section on the flange that preferentially folds as the flange passes through the fistula. Retention of the flange in a gel cap is improved by forming a foldable flap extension on a cylindrical flange. A tubular tool for insertion of a tubular voice prosthesis through a fistula is also disclosed.

### TECHNICAL FIELD

This invention relates to a prosthesis with a foldable flange which is inserted through an opening in a wall of the body such as a fistula through tracheoesophageal wall, an opening through a nasal septum or a puncture through a tympanic membrane. More particularly, this invention relates to a type of voice prosthesis developed for patients who do not choose to or cannot themselves insert the voice prosthesis due to physical, mental or other limitations. The voice prosthesis has an esophageal retention flange which could damage or cause discomfort upon insertion through the fistula if not folded, and must be inserted by a health care professional.

### BACKGROUND OF THE INVENTION

There are several options for restoring speech to patients who have had their larynx removed. One procedure is to surgically create a puncture or fistula between the trachea and the esophagus. A tracheoesophageal voice prosthesis containing a one-way valve such as a BLOM-SINGER® voice prosthesis is inserted into the tracheoesophageal fistula. The one-way valve protects the airway during swallowing but opens under positive pressure. The voice prosthesis, thus, permits a patient to divert air from the lungs into the esophagus and out through the mouth. Voice is produced by airflow induced vibration of the tissues of the upper part of the esophagus, and pharynx or throat.

The voice prosthesis maintains fistula patency, transfers air from the trachea to the esophagus for voice production and prevents esophageal leakage into the trachea during swallowing. However, the prosthesis being in contact with moisture in a hot, dark environment is subject to growth of commonly found yeast, typically Candida Albicans on the valve and the retaining flange. The growth of yeast can interfere with function of the valve resulting in leakage of swallowed matter through the incompetent valve.

The current low pressure voice prosthesis can be removed by the patient every few days and can be replaced with a clean prosthesis. The removed prosthesis can be soaked in hydrogen peroxide to prevent yeast formation in/on the valve and flange. Some patients however, have difficulty managing frequent removal and reinsertion of the prosthesis. Others, who are physically limited are not able to remove, sterilize, or reinsert the prosthesis. Furthermore, the fistula is tender and is bleeding immediately after surgery.

An extended wear, indwelling, low pressure voice prosthesis has been developed that can remain in place in the tracheoesophageal fistula for a considerable period of time, depending on the patient and conditions of use. Trips to a health care specialist to remove and replace the prosthesis are less frequent resulting in greater convenience and lower cost to the patient.

The flange or collar that rests against the tracheoesophageal wall is strengthened by increasing the thickness and/or diameter of the flange. The stronger and larger collar reduces possibility of dislodgment of the prosthesis during a coughing or sneezing episode.

The indwelling voice prosthesis is preferred since it is removed less frequently, requires less cleaning and less trauma since it is reinserted less frequently. The prosthesis contains relatively large, thick and wide flanges in order to retain the prosthesis and to seal the tracheoesophageal puncture on both sides of the tracheoesophageal wall. The tracheoesophageal puncture is small in relation to the flanges, making insertion difficult, uncomfortable, and painful for the patient.

### STATEMENT OF THE PRIOR ART

Vural et al (Journal of the American Academy of Otolarynyology 1999 p. 599) suggested that forming a wedge shaped notch in the tracheal flange facilitates insertion of a voice prosthesis through a stoma due to the increased pliability of the notched flange. The notched flange also allows the flange to fold in an insertion device such as a gel cap. However, the notched flange does not seal to the tissues and the cut tends to propagate in the elastomeric flange. Cutting a wedge out of the flange to form a notch creates rough edges that rub against contacting tissues resulting in tissue damage, irritation, and granulation.

Bruce in U.S. Pat. No. 4,695,275 discloses a middle ear ventilation or drain tube in which two flexible arms extend laterally from the end of the tube. The arms are notched at their juncture so that they can be squeezed together rearwardly during insertion of the device in tympanic opening. The arms do not form a continuous seal with the wall surrounding the opening.

### STATEMENT OF THE INVENTION

An improved prosthesis is provided in accordance with the invention that facilitates insertion and removal of the device from a puncture without compromising adjacent tissue or the sealing and retention function of the flange. The prosthesis is formed of a solid or tubular elastomeric body having at least one flange having improved foldability mounted on the body.

Foldable flanges according to the invention can be folded toward or away from the cylindrical body to which they are attached. The flanges will fold in either direction whether located at the end of the body or displaced inward from the end of the body of the prosthesis.

The foldability of at least one of the flanges is increased by forming at least one reduced thickness section in at least one of the flanges. Preferably there are at least 2 reduced thickness sections that are on opposed sides of the flange. At least one of the reduced thickness sections extends from the outer edge of the flange toward the inner edge of the flange, generally at least about 30% of the distance between the inner and outer edges. The reduced thickness sections can be a simple fold line but preferably have a polygonal shape so that the vane-like sections can elevate and fold over the remaining petal-like sections. Fold lines, cuts and slots generally are radially directed toward the center of the cylindrical body of the device.

The reduced thickness sections are preferably formed in the outside surface of the flanges such that the inside surface of the flanges are flat and continuous and form a safe, non-irritating and competent seal with the seating tissue surfaces though foldability can also be provided by forming zero thickness cuts or slots in the flange.

A voice prosthesis of the invention has a tubular elastomeric body having a valve mounted adjacent one end of the body. Annular flanges are mounted at the distal and proximal ends of the body. The device optionally contains a rigid cartridge insert to support the tubular portion of the body and to act as a seat for the valve.

The voice prosthesis of the invention can be inserted immediately after surgery even though the fistula is tender and bleeding. After the fistula heals, scar tissue and granulation form which can cause leakage during use and can be disrupted during removal and reinsertion of a voice prosthesis. The foldable flange seals effectively even to granulated and scar tissue and does not disrupt this tissue during removal and/or reinsertion of the prosthesis in the fistula.

The modified flange readily enters a gel cap or a tubular insertion device. However, it is sometime difficult to insert the flange into a gel cap without aid of a tool which is burdensome and cumbersome to the clinician. The device tends to spontaneously release from a gel cap.

Insertion and retention of a foldable flange according to this invention in a gel cap is facilitated by elongating a portion of the flange to form a triangular flap. The flap extension preferably contains a central reduced thickness fold line. The extension flap is first folded and inserted into a gel cap, followed by the rounded end, using a shoe horn-type device, if needed. Insertion of the easily folded, gel cap retained, flanged prosthesis into the puncture is easy and atraumatic.

One of the insertion devices on the market has a slotted spreadable forward end. The insertion tool spreads the fistula from 22 French to about 43 French to deploy the esophageal flange and then the insertion tool is pulled back to seat the tracheal flange. The spreading action is very painful. The hard, sharp spreadable fingers can cut or disrupt the surrounding tissues causing trauma and bleeding. The prosthetic device can be over inserted into the esophagus.

However use of a voice prosthesis with foldable flanges according to the invention with the push rod insertion device results in reduced trauma and easier insertion of the prosthesis. The flanges can be rounded and both flanges can be provided with the thick-thin foldable structure. Less force is required to insert and remove the device from a fistula. The flanges, insertion tools and gel caps can be smaller in diameter.

Another advantage of the foldable flange used in the push rod spreadable tip inserter is that there is less spreading of the fistula during insertion of the voice prosthesis device.

The prosthesis with foldable flange can also be used to plug or aerate openings in other body walls such as a nasal septum or a tympanic membrane.

The foldable flange of the invention forms a continuous seal and the thick and thin vanes forming the flange interleave when folded.

These and many other features and attendant advantages of the invention will become apparent as the invention becomes better understood by reference to the following detailed description when considered in conjunction with the accompanying drawings.

### BRIEF DESCRIPTION OF THE DRAWINGS

FIG. 1 is a side view in elevation of a voice prosthesis with a thick-thin four equal segment flange in accordance with the invention.

FIG. 2 is a side view in elevation of a voice prosthesis with a 6 segment flange.

FIG. 3 is a side view in elevation of a voice prosthesis flange with 2 wide diverging segments and 2 narrow converging segments.

FIG. 4 is a side view in elevation of a voice prosthesis flange with two opposed thick rectangular segments.

FIG. 5 is a side view in elevation of a voice prosthesis flange with a triangular extension.

FIG. 6 is a side view in elevation of another embodiment of the extended voice prosthesis illustrated in FIG. 5.

FIG. 7 is a side view in elevation of the flange similar to FIG. 6 with a fold line in one of the thick segments.

FIG. 8 is a side view in elevation of a flange with extension having a fold line in the extension and a single thin segment;

FIG. 9 is a front view in elevation of a voice prosthesis flange with extension having 2 thin segments and two thick segments with a decreased thickness fold line.

FIG. 10 is a view in section through line 10-10 of FIG. 9.

FIG. 11 is a view partially in section illustrating the 4 segment flange of FIG. 1 inserted into a gel cap.

FIG. 12 is a view in section taken along line 13-13 of FIG. 11;

FIG. 13 is a view partially in section showing the prosthesis of FIG. 1 inside an inserter tube.

FIG. 14 is a front view in elevation of a septum plug in which the flange contains cuts;

FIG. 15 is a view in section taken along line 15-15 of FIG. 14;

FIG. 16 is a front view in elevation of a prosthesis for draining a tympanic in which the flange contains slots; and

FIG. 17 is a view in section taken along line 17-17 of FIG. 16.

### DETAILED DESCRIPTION OF THE INVENTION

The voice prosthesis valve can be formed of a tubular elastomeric body having at least one end flange. The valve can be an elastomeric disc which opens under positive pressure and seats when the pressure is removed. The valve could also be a spring loaded ball which opens under positive pressure and returns to its seat when the pressure terminates. The seat for either valve can include a rigid cartridge placed within the tubular body.

The body of the voice prosthesis can be manufactured of medical grade silicone elastomer or polyurethane. The cartridge valve or ball housing can be made of a thermoplastic fluoropolymer. The prosthesis can also contain a pattern of radiopaque tantalum dots on the esophageal flange, to assist the clinician with radiographic placement verification if desired or direct visual confirmation of esophageal flange development when the valve is manufactured of a transparent material.

The thin section of the flange can contain a cut or slot but preferably has a minimum thickness of about 0.001 inches, preferably 0.005 inches in order to seal to the tracheoesophageal wall. Flanges with thin sections having a thickness from 0.10 to 0.20 inches perform satisfactorily. The thicker sections-generally are 1.5 to 3 times as thick as the thin sections in order to retain shape as the thin sections fold. The suitable thick sections have a thickness from 0.025 to 0.050 inches, generally from 0.03 to 0.05 inches.

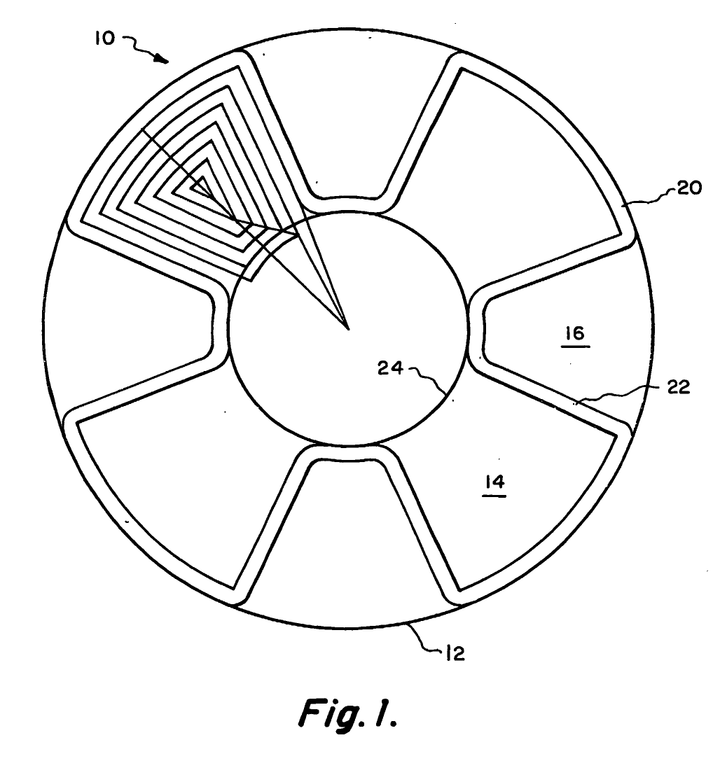

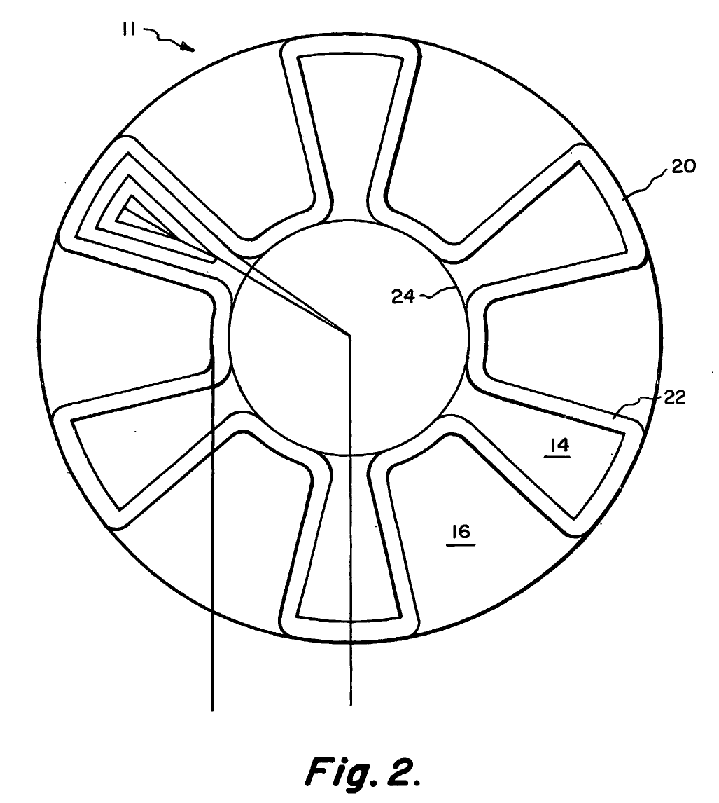

Referring now to FIG. 1, a voice prosthesis device 10 contains a flange 12 having 4 identical, evenly spaced, 4-sided segments 14 and 4 thin segments 16. All the segments 14, 16 have a wide outer edge 20, converging side edges 22 and a narrow inner edge 24. The prosthesis 11 illustrated in FIG. 2 contains 6 thick segments 14 and 6 thick segments 16.

The flange 30 illustrated in FIG. 3 has 2 thick segments 32 with wide outer edges 34 and converging sides 33 and 2 thick segments 37 with narrow outer edges 36 and diverging side edges 35. The segments 33, 37 have concave faces 39. Thin triangular segments 31 are disposed between the thick segments 32, 37.

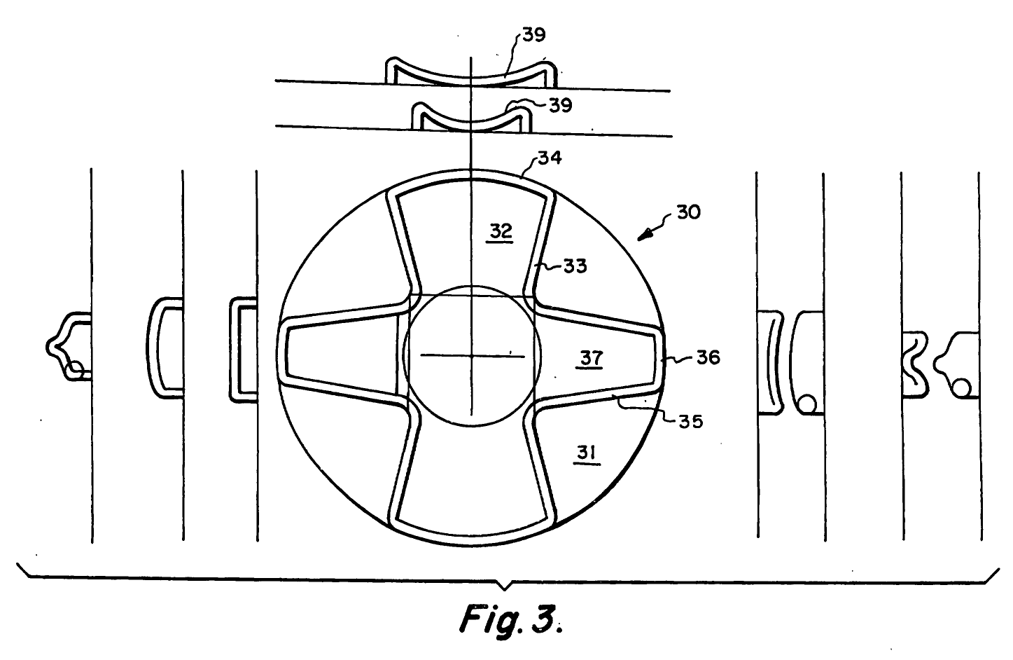

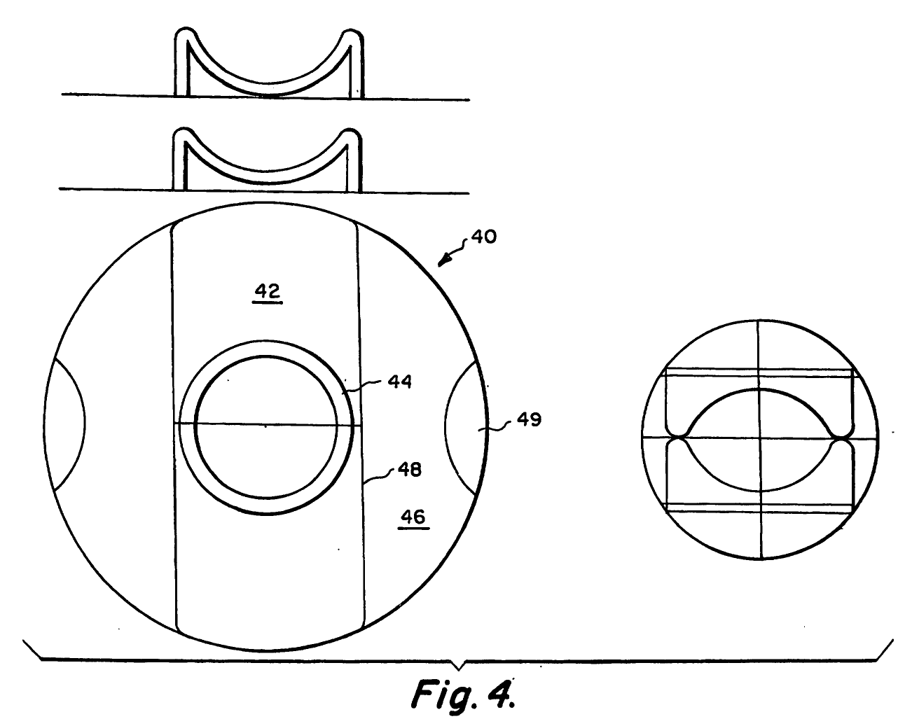

The flange 40 illustrated in FIG. 4 includes 2 opposed concave rectangular segments 42 on each side of a hood 44. A curved, circular thin segment 46 is present on the common side edges 48 of the segments 42. Thickened knobs 49 may be formed at the outer edge of the thin segments 46 for stabilizing and reinforcing the segments 46.

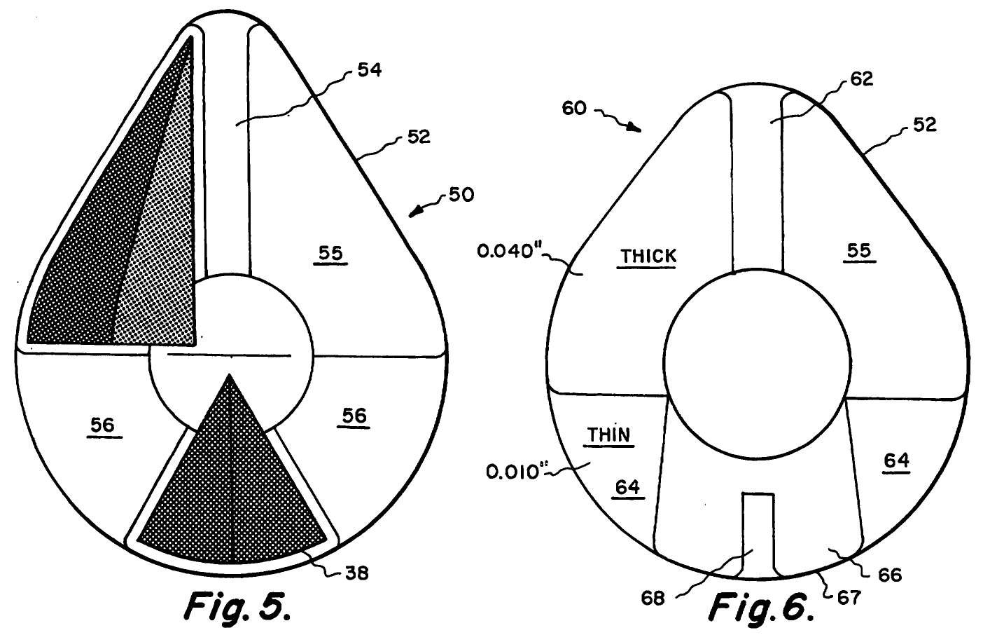

FIG. 5 illustrates a flange 50 having an extension 52 having a radial fold line 54 between thick segments 55 and two thin segments 56 on each side of a thick segment 58.

In the flange 60 shown in FIG. 6, the thick extension 52 has a wider thin fold line 62. Thin segments 64 are disposed on each side of thick rectangular segment 66 which can contain a partial, thin fold line 68 extending radially from the edge 67 of the flange toward the center of the flange.

The flange 70 shown in FIG. 7 is similar to the flange of FIG. 6 except that the rectangular segment 72 does not have the partial fold line 68 as illustrated in FIG. 6.

The flange 80 illustrated in FIG. 8 has one triangular thin segment 82 and a thin fold line 84 in the extension portion 84. The remainder of the flange has the same thickness.

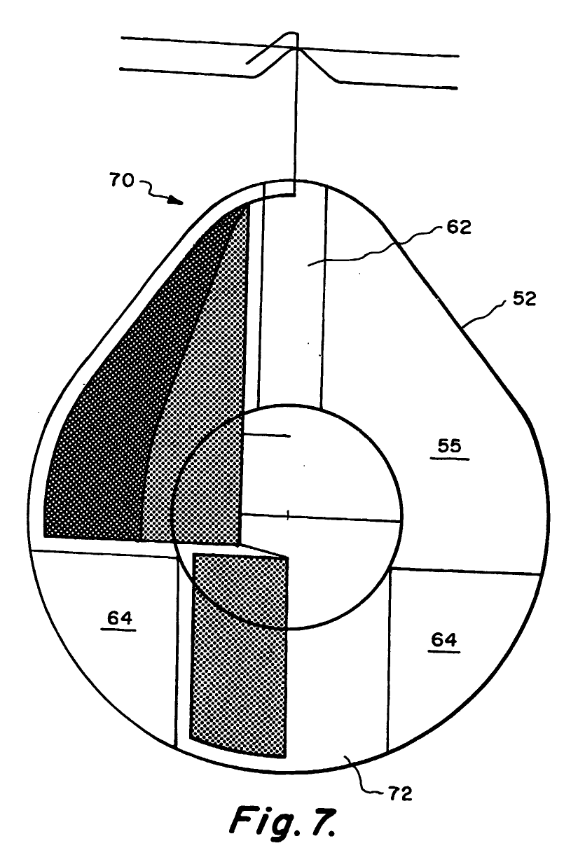

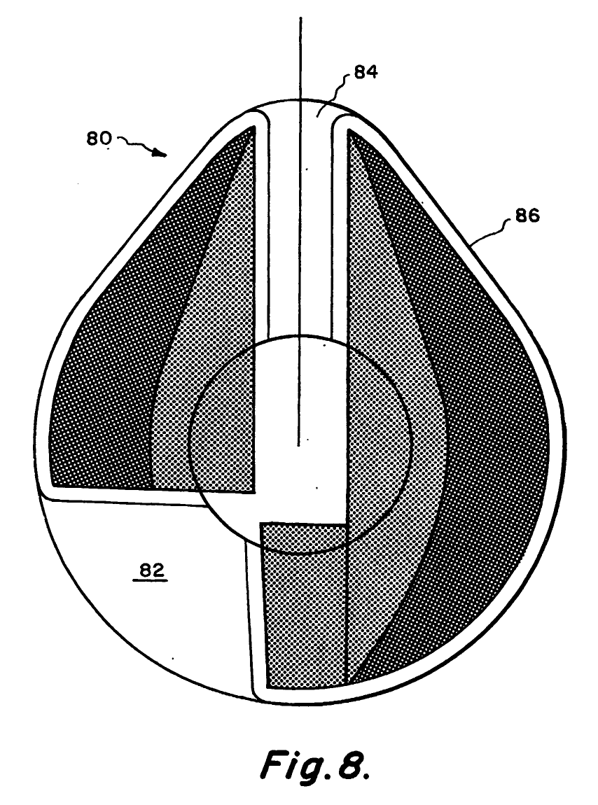

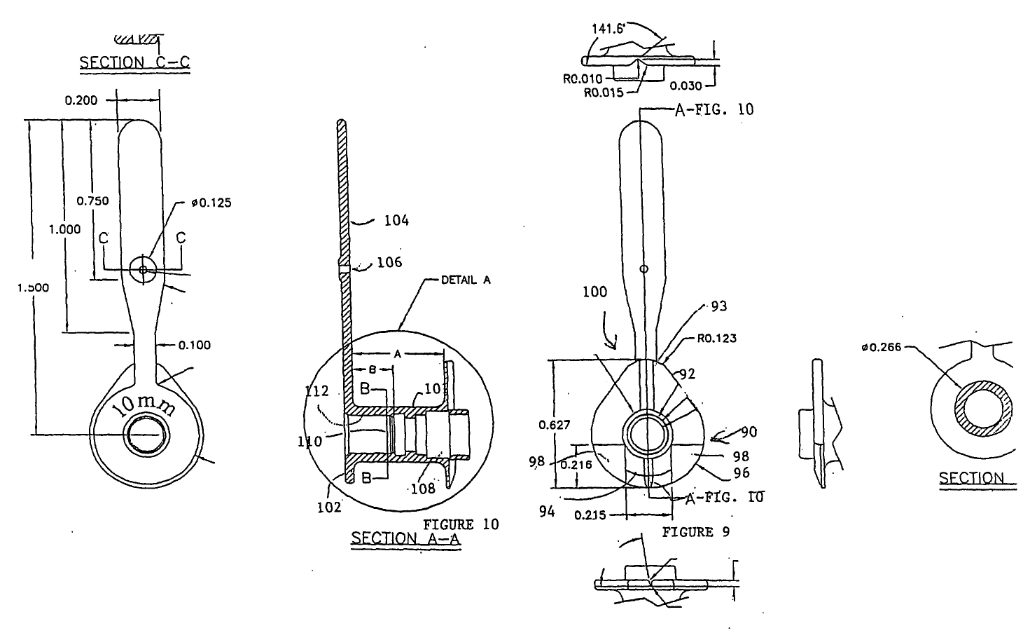

The flange 90 illustrated in FIG. 9 has a thick extension 92 with a wide folding groove 93, a thick rectangular segment 94 with a narrow groove 96 and two thin triangular segments 98 on each side of the segment 94.

Referring to FIGS. 9 and 10, a voice prosthesis 100 has a cylindrical soft body 101 containing a cylindrical tracheal flange 102 connected to a tab 104 having an aperture 106 for connection to a knob on an inserter tool, not shown. A hard cartridge 108 can be present in the body 100. A valve, not shown, can be hingedly mounted at the proximal end of the cartridge 108.

Referring now to FIGS. 14 & 15 a septal button 201 is illustrated comprised of a solid rod 203 connected to a proximal flange 205 and a distal foldable flange 206. The flange 206 can have cuts 200 forming the reducing thickness sections.

The tympanic membrane drain 220 illustrated in FIGS. 16-17 has a slot 202 forming the reduced thickness section.

The reduced thickness section can have zero thickness as shown such as a cut 200 as shown in FIGS. 14 and 15 or a slot 202 as shown in FIG. 16 and 17. The reduced thickness fold sections such as the thin, vein-like sections previously illustrated or the cut 200 or slot 202 extend inwardly from the outer edge 204 of the flanges 206, 208 at least 30% usually 50% of the distance from the outer edge 204 to the inner edge 210 of the flanges 206, 208. The reduced thickness sections usually extend no more than about 80% of the distance from the outer edge 204 to the inner edge 210 of the flange 206, 208 which is the outer surface of the central body. There are usually at least 2 reduced thickness sections and usually no more than 8. The slots 202 can have a rounded inner terminus 220 which minimizes tearing of the flange. The width of the slots 202 is as narrow as possible to minimize leakage of body fluids, generally from 0.1 inch to 30 degrees in width.

Referring now to FIGS. 11 and 12 when the device 100 is inserted into a gel cap 120, the thin segments 16 crease and form folds 122 while the thick segments 14 retain their shape.

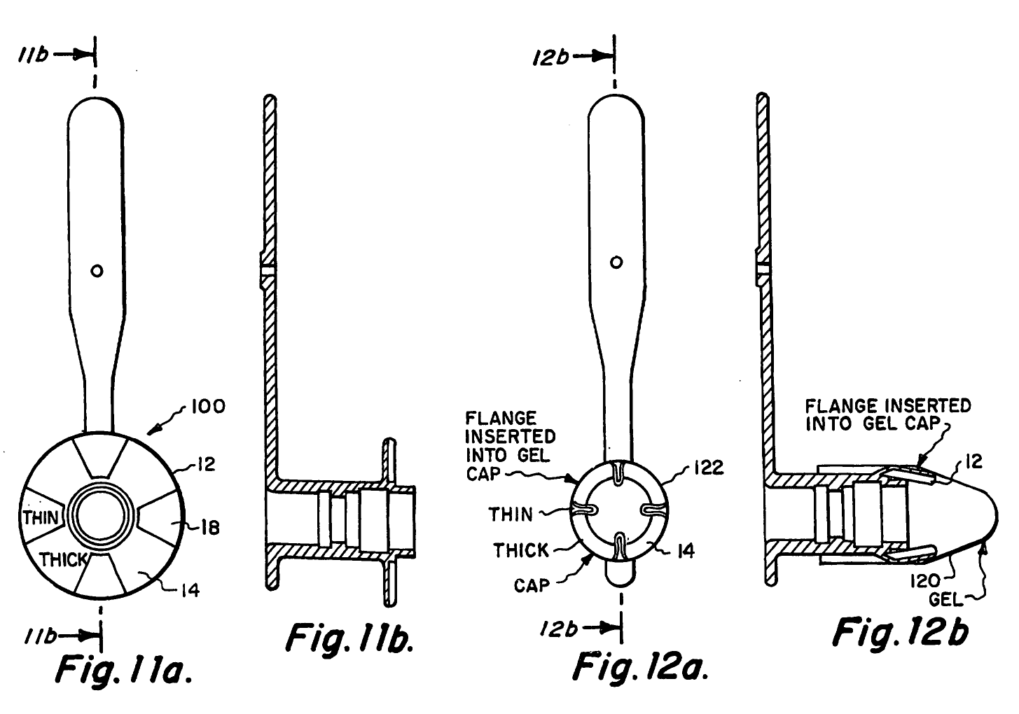

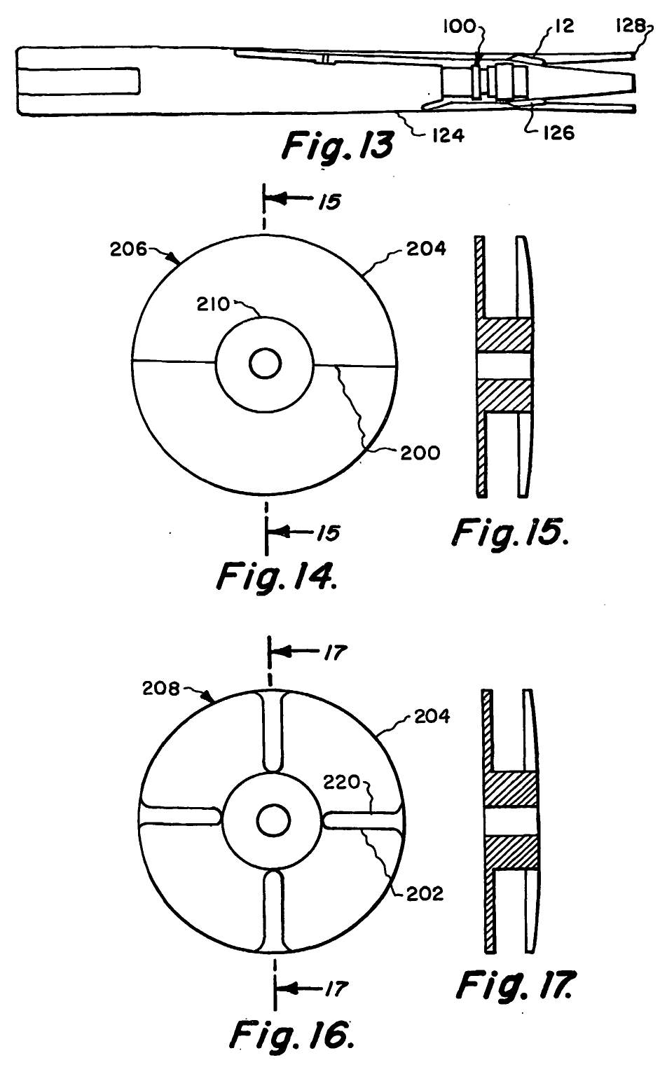

When the flange 12 on a prosthesis 100 is placed inside an inserter tube 124 the flanges fold upwardly at 126 and spread as shown in FIG. 13. The flanges deploy into a flat configuration as they leave the spreaded fingers 128 on the inserter 124.

The voice prosthesis is inserted by first placing the folded, proximal flange of the device inside the conical gel cap, then mounting the device on the end of the inserter tool. The clinician inserts the voice prosthesis into the tracheoesophageal puncture. When the gel cap dissolves, the flange unfolds to its proper configuration inside the esophagus against its anterior wall tissue surface. The device is maintained in position by the retention flange inside the esophagus as well as by the flange on the tracheal side of the puncture. The inside surface of the flanges that are in contact with tissue preferably have a smooth surface to minimize irritation.

It is to be realized that only preferred embodiments of the invention have been described and that numerous substitutions, modifications and alternations are permissible without departing from the spirit and scope of the invention as defined in the following claims.

1. A voice prosthesis for insertion into a fistula opening in a tracheoesophageal wall comprising;
a tubular elastomeric body connecting a first end to a second end of the body and having a central passage between said ends;
a foldable flange attached to the body and surrounding said body, said flange containing at least one reduced thickness section to facilitate folding of the flange.

2. A prosthesis according to claim 1 in which the flange has an outer edge and an inner edge adjacent said body and the reduced thickness section extends from the outer edge towards the inner edge.

3. A prosthesis according to claim 1 in which the flange has an outer facing surface and an inner facing surface and said section is formed in said outer facing surface.

4. A prosthesis according to claim 3 in which the inner facing surface, which contacts said wall, is substantially continuous.

5. A prosthesis according to claim 4 in which the reduced thickness portion has zero thickness and is selected from cuts or slots.

6. A prosthesis according to claim 2 in which the reduced thickness sections are polygonal sections and the flange contains at least 2 reduced thickness sections.

7. A prosthesis according to claim 6 in which the reduced thickness sections are opposed to each other.

8. A prosthesis according to claim 6 in which at least one of the reduced thickness sections is rectangular.

9. A prosthesis according to claim 6 in which at least one of the reduced thickness sections is trapezoidal.

10. A prosthesis according to claim 5 in which at least one of the reduced thickness sections is triangular.

11. A prosthesis according to claim 1 in which the flange is cylindrical.

12. A prosthesis according to claim 11 in which the cylindrical flange contains a flap extending from a portion of the outer perimeter edge of the flange.

13. A prosthesis according to claim 12 in which the flap extends from a widened base to a narrow terminus.

14. A prosthesis according to claim 13 in which the base includes at least 15% of the perimeter of the flange.

15. A prosthesis according to claim 12 in which the flap has a thickness greater than the reduced thickness sections.

16. A prosthesis according to claim 15 in which the flap contains a reduced thickness fold line.

17. A voice prosthesis for insertion into a fistula opening in a tracheoesophageal wall comprising;
a tubular elastomeric body connecting a first end to a second end of the body and having a central passage between said ends;
a foldable flange attached to the body and surrounding said body, said flange containing at least one reduced thickness section to facilitate folding of the flange;
the flange has an outer edge and an inner edge adjacent said body, the reduced thickness section extends from the outer edge towards the inner edge;
the reduced thickness sections are polygonal sections and, flange contains at least 2 reduced thickness sections.

18. A voice prosthesis for insertion into a fistula opening in a tracheoesophageal wall comprising;
a tubular elastomeric body connecting a first end to a second end of the body and having a central passage between said ends;
a foldable cylindrical flange attached to the body and surrounding said body, said flange containing at least one reduced thickness section to facilitate folding of the flange; and
the cylindrical flange contains a flap extending from a portion of the outer perimeter edge of the flange; and
the flap having a thickness greater than the reduced thickness sections;
a prosthesis according claim 18 in which the opening is a fistula in a tracheoesophageal wall and said prosthesis is a voice prosthesis.

19. A prosthesis according to claim 1 in which the opening is in a tympanic membrane and said prosthesis is a tympanic drain.

20. Cancel

21. A prosthesis according to claim 2 in which the reduced thickness sections extend inwardly from the outer edge of the flange at least 30 percent of the distance between the outer edge of the flange and the outer edge of the body of the prosthesis.

22. A septal plug prosthesis for insertion into an opening in a nasal septum wall comprising;
a tubular elastomeric body not having a central passage connecting a first end to a second end of the body;
a foldable flange attached to the body and surrounding said body, said flange containing at least one reduced thickness section to facilitate folding of the flange.

23. A prosthesis according to claim 1 in which the flange contains from 2-8 reduced thickness sections.

24. A prosthesis according to claim 22 in which the reduced thickness sections are from 0.1 and to 30 degree in width.

25. A method of inserting a flange attached to a hollow tubular, elastomeric voice prosthesis through a fistula in a tracheoesophageal wall, said flange having at least one preferentially foldable section thinner than the remainder of the flange, comprising the steps of;
folding the flange at said section away from the tubular body;
restraining the folded flange in a gel cap or other retainer or fixture made of a substance that dissolves in fluids;
inserting the folded flange through said fistula; and
unfolding the flange so that it contacts said wall.

26. Cancel

27. Cancel

28. A method of inserting a flange attached to a hollow tubular, elastomeric voice prosthesis through a fistula in a tracheoesophageal wall, said flange having at least one preferentially foldable section thinner than the remainder of the flange comprising the steps of:
folding the flange at said section;
restraining the folded flange in a hollow tubular insertion device;
inserting the folded flange through said fistula; and
unfolding the flange so that it contacts said wall.

29. Cancel

30. A method according to claim 28 in which the flange has a foldable flap extending from the edge of the cylindrical flange.
